(19) United States
(12) Patent Application Publication
(10) Pub. No.: US 2002/0133084 A1
(43) Pub. Date: Sep. 19, 2002

(54) Device for measuring blood flow to the brain

(76) Inventor: David L. Wilson, Redding, CA (US)

Correspondence Address: THEODORE J. BIELEN, JR., Bielen, Lampe & Thoeming, 1990 N. California Blvd., Suite 720, Walnut Creek, CA, 94596, US

(21) Appl. No.: 09/791,972

(22) Filed: Feb. 22, 2001

(51) Int. Cl. A61B 5/02

(52) U.S. Cl. 600/504; 600/549

(57) ABSTRACT

A device for measuring the relative blood flow rates between the right and left hemispheres of the brain utilizing a first sensor adapted to lie in the vicinity of the right ear of a mammalian subject. The first sensor generates a first signal representing the temperature of the right ear. Likewise, a second sensor is adapted to lie in the vicinity of the left ear and measure the temperature of the left ear. A signal is generated by the second sensor representing such temperature measurement. A monitor simultaneously acquires the first and second signals and the quantitative difference between the first and second signals is determined.

### BACKGROUND OF THE INVENTION

The present invention relates to a device for measuring relative blood flow between the right and left hemispheres of the brain of a mammalian subject, such as a human.

It has been recognized that the left and right sides of the mammalian brain perform different functions and are associated with different tasks. In general, left-brain hemispheres are dominant in right-handed individuals. This is especially true in humans where English was learned by the subject as a first language. The left hemisphere of the brain is specialized for symbolic representation and is associated with spoken and written language, number skills, logical and linear reasoning, fine motor control, and the positive emotions of pleasure, happiness, and joy.

The right hemisphere of the brain is dominant in left-handed individuals. In addition, the right hemisphere of the brain is associated with non-symbolic entities, direct experiences, and events that occur in the present time. The right brain is also committed to spatial skills, art, and music awareness, gross motor control, as well as the negative emotions of anger, fear, and sadness.

As a broad outline, the left hemisphere of the brain may be considered as: analytical, logical, verbal, calculating, reasoning, rational, sequential, intellectual, positive, objective, linear, and symbolic. The right hemisphere of the brain may be considered as intuitive, emotional, non-verbal, visual, relational, holistic, subjective, non-linear, simultaneous, and spatial. More simply put, the left hemisphere of the brain may be thought of as “rational” and the right hemisphere of the brain as “emotional”. Further there is increasing evidence that the right brain is more intimately and immediately involved in the registration, recall, and reactivation of traumatic events in individuals with chronic post-traumatic stress disorder (PTSD). Emerging research has also indicated that right-sided brain activation during recall of traumatic memories is consistent with what is known of the role of the right hemisphere of the brain in evaluating emotional impact of new stimuli, and in regulating autonomic responses to such information. It is also speculated that one of the roles of the left frontal lobe of the brain is to inhibit activity of the right frontal lobe and/or the right-sided limbic structures.

In many instances, the right hemisphere of the brain is thought of as a wordless early warning system for emotional activation in response to threat. The left-brain on the other hand is considered to mediate and inhibit such response to threat in the context of symbolized and consciously understood meaning. It has been established that temperature is a measure of blood flow in biofeedback applications. It is known that the greater the blood flow to a portion of the body, such body portion will exhibit a higher temperature or be warmer. In the past, measurements to the forehead of the individual have been associated with the measurement of brain temperature.

A device for measuring the relative blood flow rates between the right and left hemispheres of the brain would be a notable instrument in determining emotional states of a mammalian subject.

### BRIEF SUMMARY OF THE INVENTION

In accordance with the present invention a device for measuring the relative blood flow rates between the right and left hemispheres of the brain for the purpose of medical analysis is herein provided.

The device of the present invention utilizes a first sensor adapted to lie in the vicinity of the right ear to determine a first temperature measurement thereat. The first sensor generates a first signal representing the first temperature measurement. In addition, a second sensor is adapted to lie in the vicinity of the left ear to determine a second temperature measurement at the left ear. The second sensor also generates a second signal representing second temperature measurement. The first and second sensors may take the form of thermisters which are located in earplugs that are capable of fitting into the right and left ears of the subject. Both right and left sensors may be connected by a headset choke which fits over the head of the user.

Means is also provided in the present invention for simultaneously monitoring the first and second signals and, thus, the temperature of the left and right ears of an individual. Such temperature measurements are an indirect measure of blood flow to the left and right hemispheres of the brain. Plotting such temperatures simultaneously and determining the difference between such temperatures provides an indication of the emotional state of the user and may be used to monitor treatment of individuals suffering from trauma such as PTSD. Such means for simultaneously monitoring the first and second signals may take the form of a temperature-measuring device which is capable of simultaneously displaying the temperature measurements as well as the difference between both temperature measurements.

It may be apparent that a novel and useful device for measuring the relative blood flow rates between the right and left hemispheres of the brain for medical analysis is herein provided.

It is therefore an object of the present invention to provide a device for measuring the relative blood flow rates between the right and left hemispheres of the brain which may be easily used on subjects to calculate the differences between such blood flow rates for the purpose of determining psychological states.

Another object of the present invention is to provide a device for measuring relative blood flow rates between the right and left hemispheres of the brain which permits a medical practitioner to monitor treatment of persons suffering from trauma.

A further object of the present invention is to provide a device for measuring relative blood flow rates between the rights and left hemispheres of the brain which may be employed to determine the effectiveness of pharmaceutical treatments of persons suffering from stress disorders.

A further object of the present invention is to provide a device for measuring relative blood flow rates between the right and left hemispheres of the brain which is simple to operate and whose results are easily interpreted by a medical practitioner.

The invention possesses other objects and advantages especially as concerns particular characteristics and features thereof which will become apparent as the specification continues.

### BRIEF DESCRIPTION OF THE SEVERAL VIEWS OF THE DRAWING

FIG. 1 is a partial side elevational view of the device of the present invention in place from the left and right ears of a person depicted schematically.

FIG. 2 is a sectional view of the probe used in measuring temperature used in the device of FIG. 1.

FIG. 3 is a schematic diagram depicting a method of obtaining data using the apparatus depicted in FIG. 1.

FIG. 4 is a graph showing typical results of measurements obtained the apparatus of FIG. 1 in humans having varying degrees of historical stress.

For a better understanding of the invention reference is made to the following detailed description of the preferred embodiments thereof which should be read with the prior described drawings.

### DETAILED DESCRIPTION OF THE PREFERRED EMBODIMENTS OF THE INVENTION

Various aspects of the present invention will evolve from the following detailed description of the preferred embodiments thereof which should be taken in conjunction with the hereinabove described drawings.

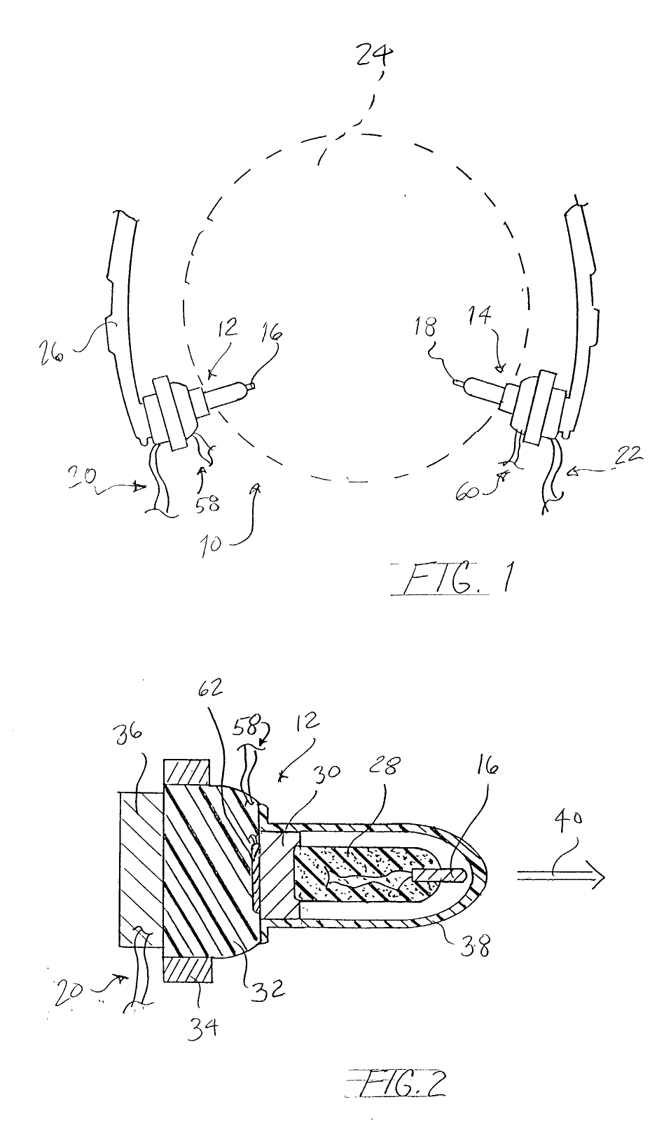

The invention as a whole is shown in the drawings by reference character 10. Device 10 includes as one of its elements a pair of sensors 12 and 14. Sensors 12 and 14 include temperature probes 16 and 18 which may be in the form of heat sensitive diodes. In any case, sensors 12 and 14 produce a signal representative of the temperature at probe 16 and 18 which is transmitted through conductors 20 and 24. Of course, such signal may be an electrical signal. Sensors 12 and 14 are intended to be placed in the left and right ears of a mammalian subject such as a human. The human head 24 is depicted schematically in FIG. 1. A headset bridge or yoke 26 connects sensors 12 and 14 and extends around the head 24 of the user.

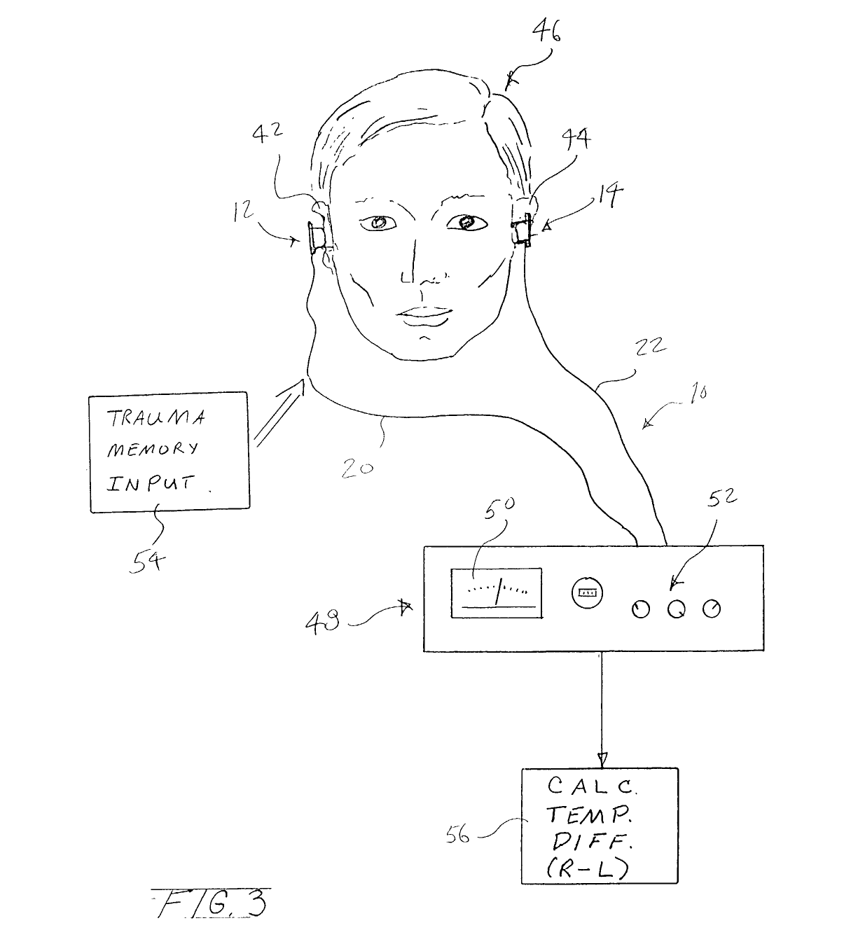

FIG. 2 represents a detail of sensor 12. However, it should be understood that sensor 14 is similarly constructed. Thermister 16 is threaded or fixed into a soft earplug 28. Earplug 20, in turn, is connected to a disk 30 which extends from member 32. A ring or cup 34 surrounds member 32. Backing element 36 fixes to member 32 and provides an exit for conductors 20. Backing element 36 also provides a seat for yoke 26 which is connected thereto in a conventional matter (not shown). Removable cover 38 friction fits over disk 30 to protect temperature probe 16. Directional arrow 40 indicates the movement of sensor 12 into an ear of a subject.

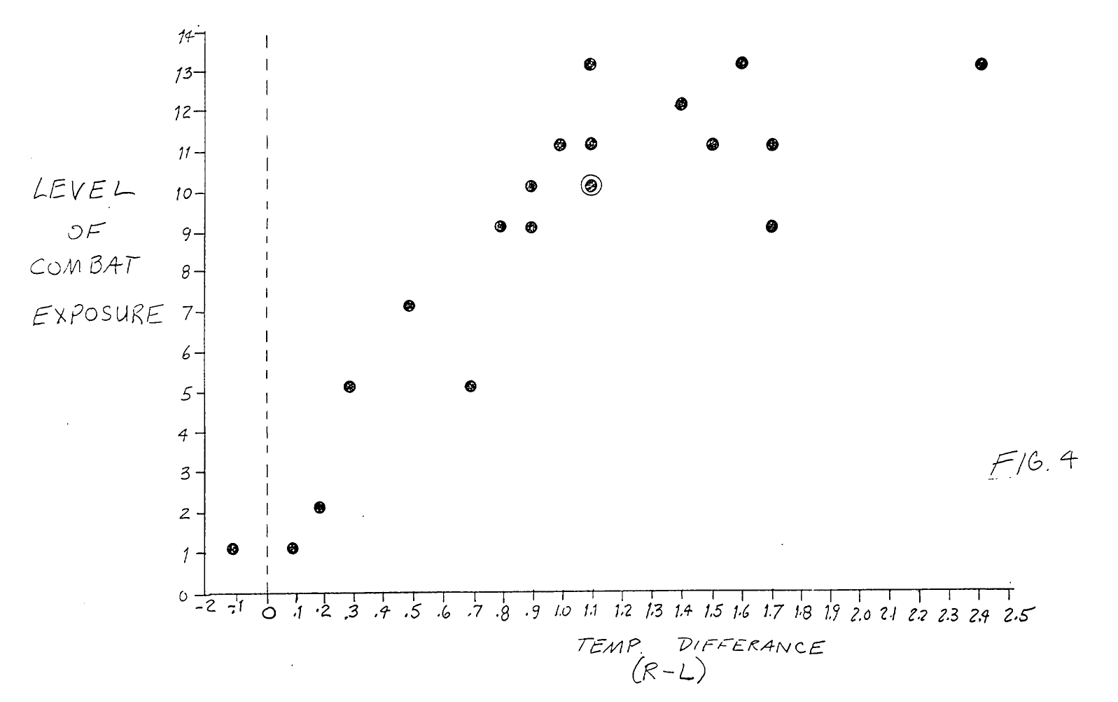

Turning to FIG. 3, it may be observed that conductors 20 and 22 send a signal representing the temperature within right ear 42 and left ear 44, respectively of subject 46. Conductors 20 and 22 feed into means 48 for simultaneously monitoring the pair of signals passing through conductors 20 and 22. Means 48 may take the form of a measuring device such as an Autogen 60 temperature-measuring device manufactured by ASI, of Berkeley, Calif. Of course other temperature measuring devices may be employed. However, in the case of the device depicted in FIG. 3, a dial 50 indicates a temperature. While controls 52 are employed to determine the scale and channel displayed on meter 50 in the present case, signals traveling through conductors 20 or 22 would be selectively displayed representing the temperature detected by sensor probes 16 or 18, respectively.

The device 10, may be used to determine psychological states of subjects such as subject 46. For example, trauma memory may be inputted into the subject 46 represented by schematic box 54. Such input may be transmitted to subject 46 through recordings, visual stimuli, or through a medical practitioner conveying information to subject 56, and the like. The temperature is recorded using device 10 in the left and right ears 42 and 44 of the subject. It is believed that such temperature measurement displayed by means 48 represents an indirect measurement of blood flow to the left and right hemispheres of the brain of subject 56. The difference in temperatures are then calculated represented by schematic box 56 to gather data which may then be studied and used for research, treatment, and the like. It should be noted that alternating sequential audio tones may be fed to ears 42 and 44 of head 24 of the patient while device 10 measures the temperature of ears 42 and 44. This audio feed process is delineated in U.S. Pat. Nos. 6,056,403 and 5,343,261, which are incorporated by reference as a whole to this application. Wire pairs 58 and 60, FIG. 1, represent such audio feed to yoke 26. Of course, each sound generating transducer, such as transducer 62, FIG. 2, would be of conventional configuration.

The following Example represents an application of device 10 but is not deemed to limit the claimed invention in any manner.

### EXAMPLE 1

A series of tests were performed in order to ascertain whether a correlation existed between a right minus left ear temperature measurements in humans and the degree of combat exposure of 20 male war veterans of the Vietnam conflict. The level of combat exposure was determined by survey with individual participants in the study ranging from merely serving in the theatre of war to being under enemy fire in combat using the apparatus described in FIG. 3. A number was assigned to each individual on a scale of 1-14, with 14 being the highest degree of exposure to wartime activities. The temperature of the right minus left ear of each patient was determined. The results are illustrated in the graph of FIG. 4. It was concluded that there was a correlation between the degree of the right minus left ear temperatures and the degree of combat exposure.

All temperatures were taken five to ten minutes after the temperature probes or thermisters 16 and 18 were placed in the ears of the subjects. This procedure was followed to obtain a good baseline for all temperature measurements. In addition, all measurements were taken twice by alternating the leads 20 and 22 between temperature sensors 12 and 14. Trauma memory input 54 was triggered by recalling memories of war experience in the 20 subjects.

While in the foregoing, embodiments of the present invention have been set forth in considerable detail for the purposes of making a complete disclosure of the invention, it may be apparent to those of skill in the art that numerous changes may be made in such detail without departing from the spirit and principles of the invention.

What is claimed is:

1. A device for measuring relative blood flow rates between the right and left hemispheres of the brain of a mammal; comprising:
a. a first sensor adapted to lie in the vicinity of the right ear to determine a first temperature measurement of at least a portion of the right ear, said first sensor generating a first signal representing said first temperature measurement;
b. a second sensor adapted to lie in the vicinity of the left ear to determine a second temperature measurement of at least a portion of the left ear, said second sensor generating a second signal representing said second temperature measurement; and
c. means for simultaneously monitoring first and second signals.

2. The device of claim 1 which additionally includes a yoke for connecting said first and second sensors.

3. The device of claim 1 in which said at least said first temperature sensor includes a temperature probe supported in an earplug.

4. The device of claim 3 in which said earplug of said first temperature sensor is further supported by an element.

5. The device of claim 3 which further comprises a cover removably held to said at least said first sensor, said cover substantially surrounding said temperature probe.
